(19) United States
(12) Plant Patent Application Publication
(10) Pub. No.: US 2024/0164233 P1
(43) Pub. Date: May 16, 2024

(54) BEGONIA PLANT NAMED 'KRHARE01'

(50) ;

(71) Applicant: KOPPE ROYALTY B.V., Putten (NL)

(72) Inventor: LUBBERTUS H. KOPPE, Putten (NL)

(73) Assignee: KOPPE ROYALTY B.V., Putten (NL)

(21) Appl. No.: 18/234,199

(22) Filed: Aug. 15, 2023

Related U.S. Application Data
(60) Provisional application No. 63/425,353, filed on Nov. 15, 2022.

(51) Int. Cl. A01H 6/18

(52) U.S. Cl. PLT/349

(57) ABSTRACT

A new and distinct cultivar of Begonia plant named ‘KRHARE01’ characterized by its broadly upright to spreading and mounded plant habit; moderately vigorous growth habit; moderately freely branching habit; dark green-colored leaves; freely flowering habit; double-type male flowers that are bright red in color and held above and beyond the foliar plane; and good postproduction longevity.

### CROSS-REFERENCED TO CLOSELY-RELATED APPLICATIONS

Title: Varieties of Begonia Plants

Inventor/Applicant: Josef Heuger

Filed: Nov. 15, 2022

Ser. No. 63/425,353

Inventor/Applicant hereby claims priority to this provisional U.S. Patent Application.

An European Community Plant Breeder's Rights application for the instant plant was filed by the Applicant/Assignee, Koppe Royalty B.V. of Putten, The Netherlands on Nov. 23, 2021, application number 2021/3013. Foreign priority is not claimed to this application.

Botanical designation: Begonia x hiemalis.

Cultivar denomination: ‘KRHARE01’.

### BACKGROUND OF THE INVENTION

The present invention relates to a new and distinct cultivar of Begonia plant, botanically known as Begonia x hiemalis, commercially referred to as an Elatior Begonia and hereinafter referred to by the name ‘KRHARE01’.

The new Begonia plant is a product of a planned breeding program conducted by the Inventor in Ermelo, The Netherlands. The objective of the breeding program is to create new freely-branching and freely-flowering Begonia plants with attractive flower color and excellent postproduction longevity.

The new Begonia plant originated from a cross-pollination made by the Inventor in December, 2017 of a proprietary selection of Begonia x tuberhybrida identified as code number KV15K2113-004, not patented, as the female, or seed, parent with a proprietary selection of Begonia socotrana identified as code number S00, not patented, as the male, or pollen, parent. The new Begonia plant was discovered and selected by the Inventor as a single flowering plant from within the progeny of the stated cross-pollination in a controlled greenhouse environment in Ermelo, The Netherlands in November, 2018.

Asexual reproduction of the new Begonia plant by terminal vegetative cuttings taken in a controlled greenhouse environment in Ermelo, The Netherlands since March, 2019 has shown that the unique features of this new Begonia plant are stable and reproduced true to type in successive generations.

### SUMMARY OF THE INVENTION

Plants of the new Begonia have not been observed under all possible combinations of environmental conditions and cultural practices.

The phenotype may vary somewhat with variations in environmental conditions such as temperature, daylength and light intensity, without, however, any variance in genotype.

The following traits have been repeatedly observed and are determined to be the unique characteristics of ‘KRHARE01’. These characteristics in combination distinguish ‘KRHARE01’ as a new and distinct Begonia plant:
1 Broadly upright to spreading and mounded plant habit.
2. Moderately vigorous growth habit.
3 Moderately freely branching habit.
4. Dark green-colored leaves.
5. Freely flowering habit.
6. Double-type male flowers that are bright red in color and held above and beyond the foliar plane.
7. Good postproduction longevity.

Plants of the new Begonia differ primarily from plants of the female parent selection in flower color as flowers of plants of the new Begonia are bright red in color whereas flowers of plants of the female parent selection are orange in color.

Plants of the new Begonia differ primarily from plants of the male parent selection in flower form as flowers of plants of the new Begonia are double-types whereas flowers of plants of the male parent selection are single-types. In addition, flowers of plants of the new Begonia are bright red in color whereas flowers of plants of the male parent selection are purplish pink in color.

Plants of the new Begonia can be compared to plants of Begonia x hiemalis ‘Baladin’, not patented. In side-by-side comparisons, plants of the new Begonia differ primarily from plants of ‘Baladin’ in flower color as plants of the new Begonia have lighter red-colored flowers than plants of ‘Baladin’.

### BRIEF DESCRIPTION OF THE PHOTOGRAPHS

The accompanying colored photographs illustrate the overall appearance of the new Begonia plant showing the colors as true as it is reasonably possible to obtain in colored reproductions of this type. Colors in the photographs may differ slightly from the color values cited in the detailed botanical description which accurately describe the colors of the new Begonia plant.

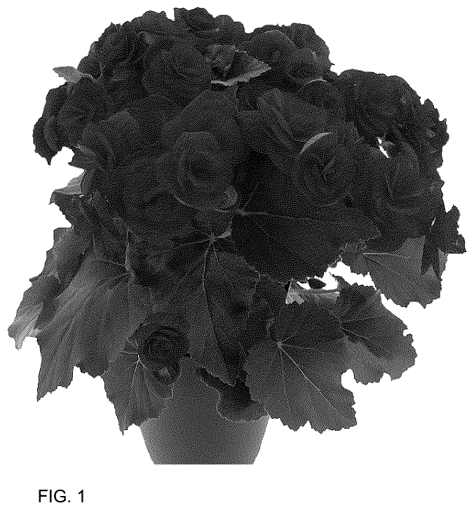

The photograph on the first sheet (FIG. 1) is a side perspective view of a typical plant of ‘KRHARE01’ grown in a container.

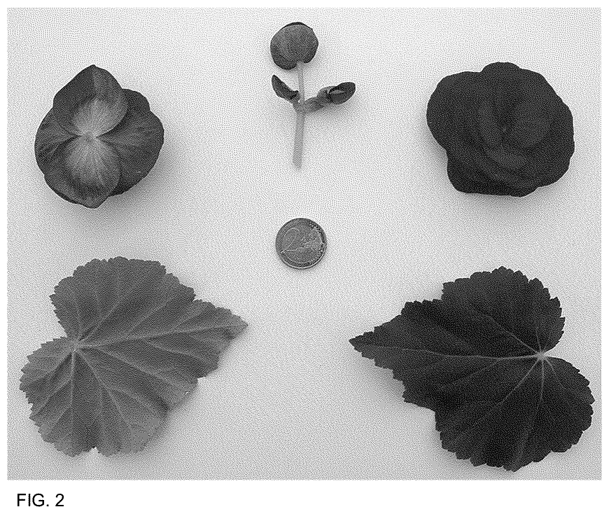

The photograph on the second sheet (FIG. 2) are close up views of the upper (right) and lower (left) surfaces of typical leaves and flowers of ‘KRHARE01’, and at the top center of the second sheet is a view of typical developing flower buds.

### DETAILED BOTANICAL DESCRIPTION

The aforementioned photographs and following observations and measurements describe plants grown during the summer in 13-cm containers in a glass-covered greenhouse in Ermelo, The Netherlands and under cultural practices typical of commercial Begonia production. During the production of the plants, day temperatures ranged from 20 C to 22 C and night temperatures ranged from 16 C to 18 C. Plants were three months old when the photographs and description were taken. In the following description, color references are made to The Royal Horticultural Society Colour Chart, 2015 Edition, except where general terms of ordinary dictionary significance are used.
Botanical classification: Begonia x hiemalis ‘KRHARE01’.
Parentage:
   Female, or seed, parent.—Proprietary selection of Begonia x tuberhybrida identified as code number KV15K2113-004, not patented.
   Male, or pollen, parent.—Proprietary selection of Begonia socotrana identified as code number S00, not patented.
Propagation:
   Type.—By terminal vegetative cuttings.
   Time to initiate roots, summer and winter.—About 20 days at temperatures about 20 C.
   Time to produce a rooted young plant, summer and winter.—About five weeks at temperatures about 20 C.
   Root description.—Fine, fibrous; typically white to orangish brown in color, actual color of the roots is dependent on substrate composition, water quality, fertilizer type and formulation, substrate temperature and physiological age of roots.
   Rooting habit.—Freely branching; medium density; to date, plants of the new Begonia have not been observed to form tubers.
Plant description:
   Plant habit and form.—Broadly upright to spreading and mounded plant habit; broadly oblong to close to spherical in overall plant shape.
   Growth habit.—Moderately vigorous growth habit and moderate growth rate; suitable for 9-cm and larger containers; under optimal environmental and cultural conditions, usually about eleven weeks from rooted cuttings are required to produce proportional plants in 13-cm containers.
   Branching habit.—Moderately freely branching with about three primary branches each with about three secondary branches developing per plant.
   Plant height, soil level to top of foliar plane.—About 26.2 cm.
   Plant height, soil level to top of floral plane.—About 29.1 cm.
   Plant width.—About 34.6 cm.
   Lateral branches.—Length: About 12.1 cm. Diameter: About 1.1 cm. Internode length: About 3 cm. Strength: Moderately strong. Aspect: Erect to about 50 degrees from vertical. Texture and luster: Smooth, glabrous; slightly glossy. Color, developing: Close to 146C. Color, developed: Close to 148A. Lenticels: Quantity: Sparse. Length: About 2 mm. Width: About 0.75 mm. Shape: Linear. Color: Close to 145B.
   Leaves.—Arrangement: Alternate, simple. Length: About 15 cm. Width: About 10.6 cm. Shape: Unequal broadly ovate. Apex: Acute to minutely apiculate. Base: Oblique, lobes free to imbricate. Margin: Crenate to dentate; slightly to moderately undulate. Texture and luster, upper surface: Smooth, glabrous; velvety; slightly glossy. Texture and luster, lower surface: Mostly smooth and glabrous with sparse pubescence along the venation; velvety; moderately glossy. Venation pattern: Pinnate. Color: Developing leaves, upper surface: Close to 137B. Developing leaves, lower surface: Close to 148B. Fully expanded leaves, upper surface: Darker than a blend of 147A and N189A; venation, close to 146B. Fully expanded leaves, lower surface: Close to 191A; venation, close to 146C. Petioles: Length: About 8.2 cm. Diameter: About 6 mm. Strength: Low, flexible. Texture and luster, upper and lower surfaces: Sparsely pubescent; slightly glossy. Color, upper surface: Close to 152B slightly tinged with close to 176C; distally, strongly tinged with close to 172A. Color, lower surface: Close to 152B. Stipules: Quantity and appearance: Two leafy stipules at the base of each leaf. Length: About 1.1 cm. Width: About 1.2 cm. Shape: Broadly ovate to close to deltoid. Apex: Broadly acute. Base: Broadly cuneate. Margin: Finely ciliate. Color, upper and lower surfaces: Close to 147D; towards the margins, close to 182D.
Flower description:
   Flower form and flowering habit.—Double-type male rotate flowers arranged in axillary cymes; to date, female flower development has not been observed on plants of the new Begonia; typically about seven flowers per cyme, numerous cymes in flower simultaneously and about 250 flowers developing per plant; flowers face upright to outwardly and are positioned above and beyond the foliar plane.
   Natural flowering season.—Plants flower continuously from the spring into the autumn in The Netherlands.
   Flower longevity.—Individual flowers last about two weeks on the plant; flowers not persistent.
   Fragrance.—None detected.
   Inflorescence height.—About 15.2 cm.
   Inflorescence diameter.—About 13.3 cm.
   Flower buds.—Length: About 1.7 cm. Diameter: Ranging from about 9 mm to 20 mm. Shape: Reniform, flattened. Texture and luster: Smooth, glabrous; slightly velvety; matte. Color: Close to 43A; towards the base, close to 180C.
   Flowers.—Diameter: About 7 cm by 7.5 cm. Depth: About 4.6 cm. Shape and type: Rotate; double. Tepals: Quantity and arrangement: About four arranged in two whorls. Length, outer whorl tepals: About 3.9 cm. Length, inner whorl tepals: About 3.6 cm. Width, outer whorl tepals: About 4.3 cm. Width, inner whorl tepals: About 4.2 cm. Shape, all tepals: Reniform. Apex, all tepals: Mostly rounded. Base, outer whorl tepals: Truncate. Base, inner whorl tepals: Cuneate. Margin, all tepals: Entire, not undulate. Texture and luster, upper surface, all tepals: Smooth, glabrous; velvety; matte. Texture and luster, lower surface, all tepals: Smooth, glabrous; velvety; slightly glossy to matte. Color, outer whorl tepals: When opening, upper surface: Close to N45B. When opening, lower surface: Close to 180B; towards the margins and apex, close to 47A. Fully opened, upper surface: Close to 46B to 46D; venation, close to 46B to 46D; color does not change with subsequent development. Fully opened, lower surface: Close to 48A and 48B; towards the margins and apex, close to 47B; venation, similar to lamina colors; color does not change with subsequent development. Color, inner whorl tepals: When opening, upper surface: Close to 46B. When opening, lower surface: Close to 45A. Fully opened, upper surface: Close to a blend of 45B and 46C; venation, close to a blend of 45B and 46C; color does not change with subsequent development. Fully opened, lower surface: Close to 45C; towards the margins and apex, close to 46B; venation, similar to lamina colors; color does not change with subsequent development. Tepaloids: Quantity and arrangement per flower: About 22 to 50 per flower arranged in about six whorls. Length: About 9 mm to 36 mm. Width: About 7 mm to 40 mm. Shape: Obovate to close to orbicular. Apex: Obtuse. Base: Cuneate. Margin: Entire; not undulate. Texture and luster, upper and lower surfaces: Smooth, glabrous; velvety; matte. Color: When opening, upper surface: Close to 45B. When opening, lower surface: Close to 46B. Fully opened, upper surface: Close to a blend of 46B and 46C; color does not change with subsequent development. Fully opened, lower surface: Close to 46C; color does not change with subsequent development.
   Flower bracts.—Quantity and arrangement: Two positioned at the top of the peduncle. Length: About 1.3 cm. Width: About 1.5 cm. Shape: Broadly ovate. Apex: Broadly acute. Base: Broadly cuneate. Margin: Finely ciliate. Texture and luster, upper and lower surfaces: Smooth, glabrous; moderately velvety; slightly glossy. Color, upper and lower surfaces: Translucent, close to 179A and 179B; towards the base, close to 146D.
   Peduncles.—Length: About 6.6 cm. Diameter: About 6 mm to 7 mm. Strength: Moderately strong. Aspect: About 30 degrees from lateral branch axis. Texture and luster: Sparsely pubescent; moderately glossy. Color: Close to 146C.
   Pedicels.—Length: About 3.2 cm. Diameter: About 2.5 mm. Strength: Moderately strong. Aspect: About 50 degrees from peduncle axis. Texture and luster: Sparsely pubescent; glossy. Color: Close to 151A strongly tinged with close to N170B.
   Reproductive organs.—To date, no stamen and pistil development has been observed on plants of the new Begonia.
   Seeds and fruits.—To date, seed and fruit development have not been observed on plants of the new Begonia.
Pathogen & pest resistance: To date, resistance to pathogens and pests common to Begonia plants has not been observed on plants of the new Begonia.
Temperature tolerance: Plants of the new Begonia have been observed to tolerate high temperatures about 35C and to be suitable for USDA Hardiness Zones 10 to 12.

It is claimed:

1. A new and distinct Begonia plant named ‘KRHARE01’ as illustrated and described.
